(19) United States
(12) Patent Application Publication
(10) Pub. No.: US 2009/0248011 A1
(43) Pub. Date: Oct. 1, 2009

(54) CHRONIC VENOUS INSUFFICIENCY TREATMENT

(76) Inventors: Edwin J. Hlavka, Minneapolis, MN (US); Michael Selzer, Wayzata, MN (US)

Correspondence Address: SCHWEGMAN, LUNDBERG & WOESSNER, P.A., P.O. BOX 2938, MINNEAPOLIS, MN, 55402, US

(21) Appl. No.: 12/396,178

(22) Filed: Mar. 2, 2009

Related U.S. Application Data
(60) Provisional application No. 61/067,475, filed on Feb. 28, 2008.

(51) Int. Cl. A61B 18/14; A61F 7/00

(52) U.S. Cl. 606/41; 607/105

(57) ABSTRACT

A first tissue region can be ablated after pre-cooling a nearby second tissue region to inhibit damage to the second tissue associated with the ablation of the first tissue.

### CLAIM OF PRIORITY

This patent application claims the benefit of priority, under 35 U.S.C. Section 119(e), to Hlavka et al. U.S. Provisional Patent Application Ser. No. 61/067,475, entitled “METHODS AND DEVICES FOR TREATING CHRONIC VENOUS INSUFFICIENCY,” filed on Feb. 28, 2008 (Attorney Docket No. 2802.008PRV), hereby incorporated by reference in its entirety.

### TECHNICAL FIELD

This document pertains generally to medical devices, and more particularly, but not by way of limitation, to chronic venous insufficiency treatment.

### BACKGROUND

RF ablation of superficial veins in a leg can be used to treat chronic venous insufficiency. Typically, tumescent anesthesia is provided to a target area. Tumescent anesthesia is a technique for delivering local anesthesia to a target area, and generally includes providing a large volume of diluted lidocaine and epinephrine to the target area, causing the target area to become swollen and firm (or tumescent). Following the tumescent anesthesia, a catheter-based system can be introduced into one of the superficial veins, and energy can be applied, using the catheter, to heat and injure the vein. The applied energy can result in initial shrinking or occlusion of the vein, which, over time, can result in a healing response that can completely obliterate the vein.

### OVERVIEW

A first tissue portion can be ablated after pre-cooling a nearby second tissue portion to inhibit damage to the second tissue associated with the ablation of the first tissue. In certain examples, the pre-cooling can protect the second tissue from thermal injury, or from pain associated from thermal injury.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1-3 illustrate generally examples of systems including an elongated tubular body, an expandable member, and an ablation energy source.

FIG. 4 illustrates generally an example of a system including an actuator.

FIG. 5 illustrates generally an example of a method including pre-cooling second tissue at a first time and ablating first tissue at a second time.

FIGS. 6A-6G illustrate generally examples of pre-cooling and ablating at least a portion of a great saphenous vein.

FIGS. 7A-7F illustrate generally examples of a relationship between temperature and distance for a vessel and surrounding tissue during pre-cooling and ablation.

### DETAILED DESCRIPTION

A human venous system in a leg can be divided into two subsystems, the deep system and the superficial system. The deep system is generally located deep to the fascial layers of the leg, the fascia lata, and deep fascia of the leg. Conversely, the superficial system is generally located external to these fascial layers. Each subsystem in the human venous system includes a series of one-way valves within the vein that normally prevent blood from flowing downward (e.g., away from the heart). Muscular action (e.g., by walking) can squeeze on the veins and “pump” blood up and toward the heart.

The superficial system, while highly variable and branching, includes the main portion of the Great Saphenous Vein, which runs from the anterior ankle to the groin. At the groin, the Great Saphenous Vein can join a femoral vein and the Small Saphenous Vein, running from the posterior ankle to the back of the knee where it dives deep to the fascia.

In certain examples, the deep system and the superficial system connect at the saphenous opening. The deep system and the superficial system can also communicate using a series of so-called perforator veins including short vessels that penetrate the fascial layers. The perforator veins also commonly have one-way valves that can direct blood flow from the superficial system to the deep system.

While the saphenous veins can provide a physiologic function, there is generally enough redundancy in the human vasculature that it is not absolutely necessary for a normal, healthy lifestyle. In fact, the saphenous vein is often harvested for use as a graft for use in other places in the body (e.g., during coronary arterial bypass grafting (CABG) procedures).

### Chronic Venous Insufficiency

In certain individuals, a constant pressure of blood within the venous system (e.g., exacerbated by long periods of standing, obesity, the extra blood volume associated with childbearing, or one or more other factors) can dilate the saphenous vein at one or more locations along its length. In certain examples, once the vein dilates beyond a certain point, the valve can be unable to close properly, exposing a greater length of the vein to the venous pressure. As more of the vein is exposed, the effect progressively worsens. As one valve fails, the propensity of failure in the next lower-down valve increases.

In addition to an increased pressure, the valvular incompetence can also lead to reflux or blood flow in the reverse direction (e.g., downward). In certain examples, over time, the reflux can become progressive and can affect the entire length of the saphenous system, the perforator veins, and ultimately the deep veins. When the local reflux develops into axial reflux (e.g., from groin to calf), a continuous incompetent column can exist from the heart to the ankle in the erect individual, and venous hypertension can occur.

In various examples, venous hypertension can lead to a variety of pathological conditions, including:

(1) Edema;

(2) Aching or heaviness;

(3) Leg fatigue;

(4) Itching;

(5) Skin ulcers;

(6) Skin thickening or discoloration;

(7) Varicose veins;

(8) Reticular veins;

(9) Telangiectasia veins; or

(10) One or more other pathological conditions.

In certain examples, as many as 20% of the adult population or 50% of women over the age of fifty can suffer from some form of chronic venous disease. Further, as many as six million Americans can currently be affected by skin changes due to venous insufficiency.

### Example Standard of Care

Many systems or techniques that can be used for treating chronic venous insufficiency can share certain factors in common. For example, an energy delivery device (e.g., RF electricity or laser) can be used to apply heat to the inside of the saphenous vein. The application of energy to the vein is demonstrably quite effective in eliminating blood flow acutely and of maintaining the efficacy durably over time.

In an example, an energy delivery device can be delivered such as percutaneously (e.g., like a catheter) through one or more locations on the body, such as from a location just behind and below the knee. In certain examples, a location that is behind and below the knee can be a convenient location to access the great saphenous vein. At such a location behind and below the knee, the great saphenous vein is typically quite close to the skin and can be easily visualized such as by using ultrasound or otherwise accessed such as by using one or more other Seldinger-type techniques. The energy delivery device can be advanced superiorly to just below the sapheno-femoral junction, being careful to visualize (e.g., using ultrasound) important branches, such as the superficial epigastric vein. In an example, energy can be applied a location that is well clear of the femoral vein area, which is desired not to be damaged. Then, energy can be applied to obliterate the vein and the catheter can be retracted to a new location. Using this approach, the entire length of the saphenous vein from near the groin to just below the knee can be treated.

In other examples, endovascular treatment of the saphenous vein can be completed under general anesthesia in a laboratory setting. In this example, general anesthesia can be used, such as due to pain involved with the treatment and inadvertent burns to skin, fascia, muscles, or fat. In an example, advances in anesthetics, such as tumescent anesthesia, have allowed ablation procedures to be performed in an office setting.

Tumescent anesthesia refers generally to an injection of a larger volume of agent (e.g., 5 mml of 1% xylocalne and 1 to 2 ml of 1:1000 epinephrine diluted in 1 liter of normal saline, etc.) around the exterior of the vein along its length to be treated. In certain examples, some procedures can involve 8 to 16 separate injections of anesthesia along the length of the vein to be treated.

The tumescent anesthesia provides a number of benefits, such as:

(1) Local anesthesia due to drug additives;

(2) Shrinking of a vein (e.g., vaso spasm) due to drug additives;

(3) Vein constriction due to pressure/volume of injected fluid;

(4) Separation of adjacent tissues from a vein (e.g., and possible heat conduction); and

(5) Fluid can serve as heat sink for excess heat applied to vein.

In certain examples, the benefits of tumescent anesthesia can significantly reduce the incidence of skin burns and the pain of the procedure, and can allow the anesthetic procedure to be stepped down, such as from the general anesthesia to conscious sedation that can be safely performed in an office or clinic setting.

However, tumescent anesthesia also includes several burdens. First, the volume of injected fluid can require that local anesthetics be injected first at the same location as the tumescent anesthesia will be injected. Second, the exact course of the vein in the leg is typically mapped by ultrasound so that the locations for the tumescent anesthesia can be planned. Third, each injection of tumescent anesthesia is generally performed under ultrasound guidance to ensure correct placement. Finally, an extensive series of injections typically requires that a patient's entire leg be sterilized and draped for the procedure and for the treating physician be similarly sterile.

In certain examples, in a typical tumescent anesthesia and ablation procedure taking roughly 30 minutes, only about 5 minutes is spent actually inserting and manipulating the energy delivery catheter. During this procedure, the other 25 minutes is largely devoted to sterile technique and prep, ultrasound mapping, and injection of tumescent anesthesia. Further, this 30 minute procedure time is exclusive of the significant room and patient prep which is largely driven by the extensive sterile region required.

Accordingly, it can be beneficial if tumescent anesthesia could be eliminated while still protecting adjacent tissues and the patient from excessive heat delivered to the vein. If the tumescent anesthesia step were eliminated, then, only a small sterile region at the patient's knee would be required for initial insertion of the therapeutic device.

Once the therapeutic device is inserted to its most distal location (near the patient's groin), the physician can break sterility and work without encumbrance to locate the specific area to be treated while retracting the therapeutic device.

### EXAMPLES

A first region of tissue can be ablated after pre-cooling a nearby second region tissue to inhibit damage to the second tissue associated with the ablation of the first tissue. In certain examples, the pre-cooling can protect the second tissue from thermal injury, or from pain associated from thermal injury.

In an example, a vessel (e.g., the great saphenous vein, the small saphenous vein, or one or more other veins or vessels) and tissue surrounding the vessel can be cooled (e.g., pre-cooled before ablation). In various examples, the cooling can be applied for a first period of time (a user-specified or other desired period, a preset specified threshold period, etc.), applied until a desired temperature (e.g., below a normal body temperature, etc.) is reached, or applied for one or more other periods or metric. After the cooling is applied, an ablation energy can be applied to the vessel (e.g., to an inside the vessel). In various examples, the ablation energy can be applied for a second period of time (a user-specified or other desired period, a preset threshold period, etc.), applied until a desired or specified temperature is reached, or applied for one or more other periods or metric. In an example, the cooling and the ablation energy can be configured such that the vessel can be ablated using an ablation energy, but the surrounding tissue can be protected from thermal injury or pain associated with thermal injury using the pre-cooling.

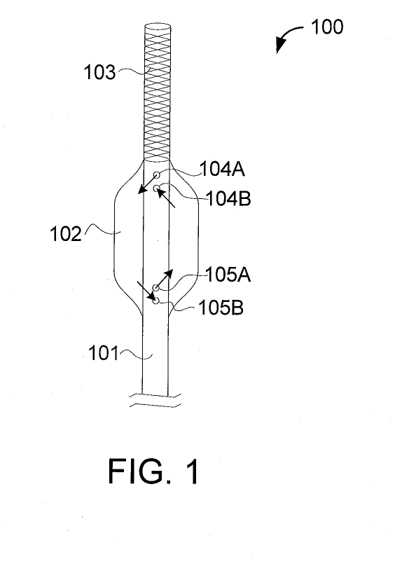

FIG. 1 illustrates generally an example of a system 100 including an elongated tubular body 101, an expandable member 102, and an ablation energy source 103.

In an example, the elongated tubular body 101 can include a catheter or other flexible medical device delivery system or tube including a lumen configured to advance one or more instruments or other matter into a vessel or vein. In this example, the vessel or vein can include the great saphenous vein, the small saphenous vein, or one or more other vessels or veins.

In an example, the expandable member 102 can include a balloon or other expandable structure configured to inflate or otherwise expand, and to receive matter, such as a gas, a liquid, or other matter. The expandable member 102 can be located near a distal end of the elongated tubular body 101. In certain examples, the expandable member 102 can be a part of the elongated tubular body 101, or the expandable member 102 can be a component separate from, but surrounding or coupled to the elongated tubular body 101.

In an example, the elongated tubular body 101 can include at least one port configured to inflate or otherwise assist in the expansion of the expandable member 102. In certain examples, the at least one port can be configured to deflate or otherwise assist in the contraction of the expandable member 102. In an example, a single port can be used to inflate or expand the expandable member 102, and then to deflate or contract the expandable member 102. In other examples, one or more input ports (e.g., a first input 104A and a second input 105A) can be used to inflate or expand the expandable member 102, and one or more output ports (e.g., a first output 104B and a second output 105B), separate from the one or more input ports, can be used to deflate or contract the expandable member 102. In an example, the one or more input ports can be denoted by the arrows pointing from the one or more ports into the expandable member 102, and the one or more output ports can be denoted by the arrows pointing from the expandable member 102 into the one or more ports.

In an example, the expandable member 102, once inflated or otherwise expanded, can be configured to contact (or be in close proximity to) a vessel wall (e.g., the wall of the great saphenous vein, the small saphenous vein, etc.) In certain examples, the expandable member 102 can be configured to block or inhibit at least a portion of flow along the vessel. In other examples, the expandable member 102 can be configured to transfer energy to the vessel wall or surrounding tissue. The transfer of energy (e.g., cooling) can be enhanced as the expandable member is placed in contact with or close in proximity to the vessel wall or tissue.

In an example, the expandable member 102 can include a cooling member configured to cool tissue surrounding the expandable member 102. In an example, the cooling member can be configured to receive a cooling matter (e.g., a cooling, cold, or chilled liquid or gas, a pressurized gas, etc.), and to cool tissue surrounding the cooling member. In an example, the cooling member can include a balloon. As the cooling member (e.g., the expandable member 102) is inflated or expanded, it can receive a larger amount or volume of the cooling matter, cooling the tissue surrounding the cooling member (e.g., a vessel wall or surrounding tissue) faster than using a smaller non-inflating volume. In other examples, if the cooling member is inflated or expanded, the cooling member can be configured to be in contact with, or be in close proximity to, the wall of the vessel, cooling the tissue surrounding the cooling member at a faster rate than using one or more other cooling members that do not expand or move to be in close proximity or in contact to the vessel wall. In other examples, the cooling member can include a member having a substantially rigid form (e.g., a member that does not expand).

In various examples, the cooling matter can include a fluid, such as saline, Ringer's lactate, a physiologic glucose solution, or one or more other fluids (including a flowable gel or other flowable substance), or the cooling matter can include a gas, such as carbon dioxide (CO₂), helium, a pressurized expanding gas (e.g., adiabatic free expansion), or one or more other gasses. In an example, the cooling matter can be chilled or can otherwise have a temperature below a normal tissue temperature (e.g., 37 degrees Celsius), below a normal room temperature (e.g., 18 to 21 degrees Celsius), or below one or more other thresholds. In an example, the cooling matter can be configured to reduce the temperature of a vessel and surrounding tissue from the normal tissue temperature (e.g., 37 degrees Celsius) to a lower temperature (e.g., 30 degrees Celsius, etc.). In an example, the reduction in temperature can be configured to reduce an affected depth of a later applied ablation energy, or can be configured to protect the tissue surrounding a target ablation site.

In an example, the ablation energy source 103 can include one or more energy sources, such as an electrode (e.g., a radiofrequency energy source) configured to provide a radiofrequency energy, a first and second electrode configured to provide (and in certain examples, receive) an electric current, an ultrasound transducer (or other sound sensor) configured to provide an ultrasound energy, or a laser (or other light source) configured to provide laser (or other light) energy (e.g., via a fiber optic conduit or other optical element or the like), or one or more other ablation energy sources configured to provide an ablation energy. In an example, the ablation energy source 103 can be located near the expandable member 102 (e.g., the cooling member), such as proximate the expandable member 102, at the distal end of the elongated tubular body 101. In various examples, the ablation energy source 103 can be coupled to the elongated tubular body 101, or the ablation energy source 103 can be included as a portion of the elongated tubular body 101.

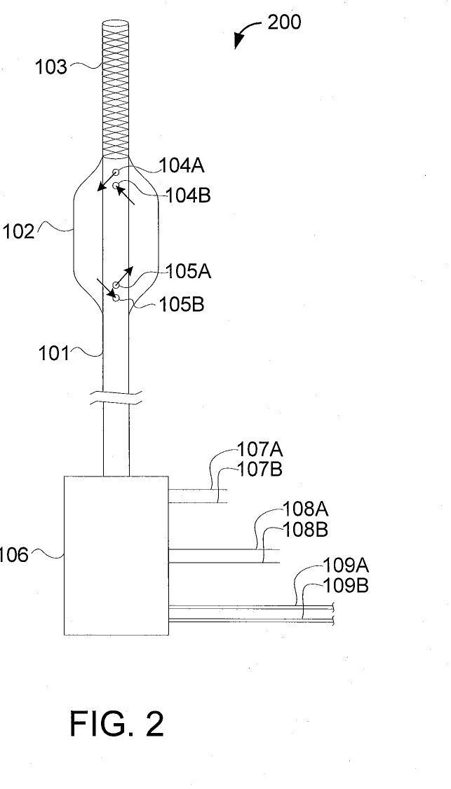

FIG. 2 illustrates generally an example of a system 200 including an elongated tubular body 101, an expandable member 102, an ablation energy source 103, and an actuator 106.

In an example, the actuator 106 (e.g., a user-actuator configured to receive instructions or input from a clinician or other user, an automated actuator, or a processor or other actuator) can be configured to actuate or control at least one of pre-cooling or ablation. In an example, the actuator 106 can be configured to control the pre-cooling and the ablation, such that the pre-cooling occurs before the ablation.

In an example, the actuator 106 can include one or more inputs or outputs configured to control the ablation energy source 103, the expandable member 102, or to send or receive other commands, controls, or other information. In an example, the actuator can include a first energy delivery channel 107A and a second energy delivery channel 107B configured to control energy delivery to the ablation energy source 103. In an example, the actuator 106 can include a cooling matter input 109A and a cooling matter output 109B configured to control delivery of the cooling matter to the expandable member 102.

In certain examples, the system 200 can include one or more temperature sensors, or other sensors configured to sense or detect a temperature or other parameter indicative of an effect of the cooling or ablation (e.g., a vessel temperature, a surrounding tissue temperature, a temperature of the expandable member 102, a temperature of the ablation energy source 103, etc.). In an example, the actuator 106 can include a first temperature channel 108A configured to receive a first temperature (e.g., a tissue temperature) and a second temperature channel 108B configured to receive a second temperature (e.g., an ablation energy source 103 temperature).

In other examples, the cooling or ablation temperatures can be estimated using a difference between the temperature of input cooling matter (e.g., a temperature of the cooling matter input 109A) and the temperature of the output cooling matter (e.g., a temperature of the cooling matter output 109B). In other examples, one or more other proxies can be used to estimate the effect of and to control the cooling or the ablation.

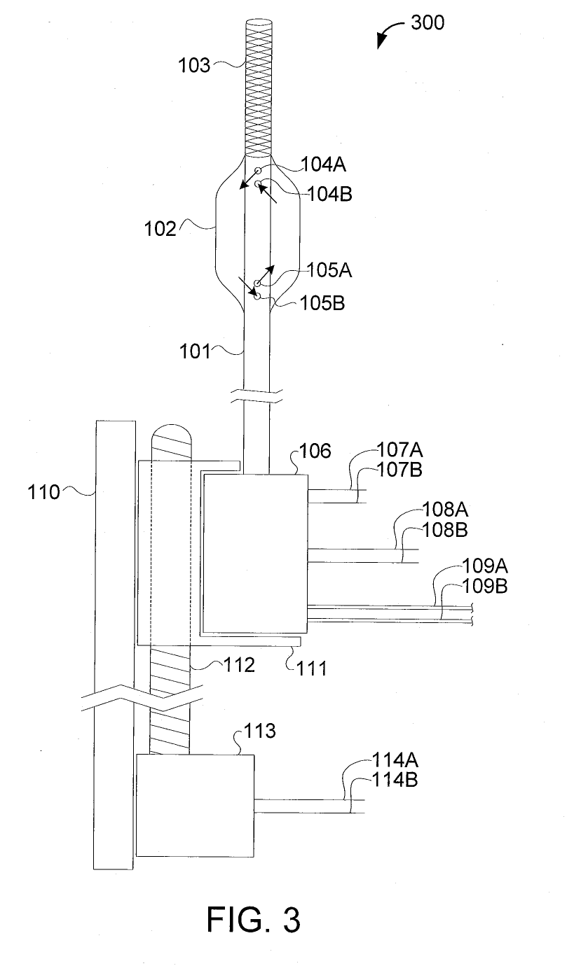

FIG. 3 illustrates generally an example of a system 300 including an elongated tubular body 101, an expandable member 102, an ablation energy source 103, an actuator 106, and a retraction module 113.

In an example, the retraction module 113 can include any mechanical actuator configured to move (e.g., insert or retract) an elongated tubular body 101. In an example, the retraction module 113 can be configured to at least partially retract the elongated tubular body 101 from a vessel. In an example, the retraction module 113 can include a retraction member 112 (e.g., a screw, a piston, or other mechanical movement actuator) coupled to at least one of the elongated tubular body 101 or the actuator 106. In an example, the actuator 106 can be coupled to the retraction member 112 using a coupler 111 configured to receive the retraction member 112. In an example, the system 300 can include a holder 110 configured to hold or guide at least one of the retraction module 113, the retraction member 112, or the coupler 111.

In an example, the retraction module 113 can include one or more inputs (e.g., a first input 114A and a second input 114B) configured to control movement (e.g., insertion or retraction) of the elongated tubular body 101. In an example, movement can be controlled by a user (e.g., a clinician), or movement can be automatic, in certain examples, assisted by one or more sensors or other automated inputs).

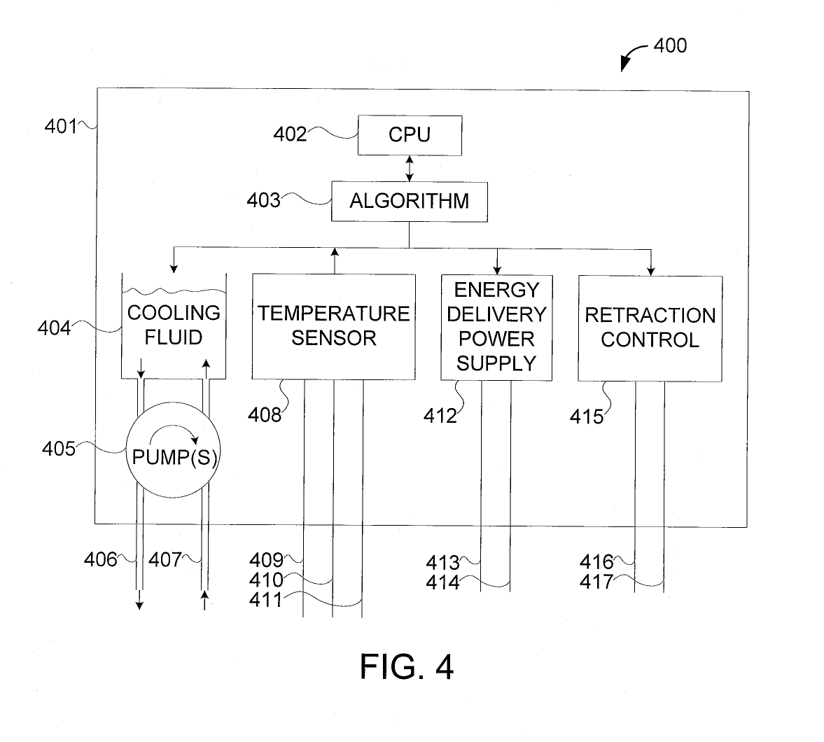

FIG. 4 illustrates generally an example of a system 400 including an actuator 401. In an example, the actuator 401 can include a central processing unit (CPU) 402, an algorithm 403 coupled to or operable on or using the CPU 402, a cooling fluid reservoir 404, a temperature sensor 408, an energy delivery power supply 412, and a retraction control 415.

In an example, the CPU 402 can include one or more actuators, controllers, or processors configured to process one or more instruction (e.g., the algorithm 403) using the one or more actuators, controllers, processors, etc. In an example, the algorithm 403 or the CPU 402 can be configured to control an amount of cooling fluid being delivered to an expandable member (e.g., the expandable member 102) from a cooling fluid reservoir 404 through a pump 405, an input 406 (e.g., to the expandable member), such as the first input 104A, and an output 407 (e.g., to the cooling fluid reservoir 404), such as the first output 105A.

In an example, the algorithm 403 or the CPU 402 can be configured to receive information from the temperature sensor 408 (e.g., using one or more temperature inputs, such as a temperature input 409, a second temperature input 410, and a third temperature input 411. In other examples, the algorithm 403 or the CPU 402 can be configured to receive one or more surrogate indications of ablation (e.g., heating) or cooling. In an example, the first temperature input 409, the second temperature input 410, and the third temperature input 411 can include temperature inputs from an ablation energy source, a first tissue, and a second tissue, respectively. In other examples, one or more other temperature inputs can be utilized or received.

In an example, the algorithm 403 or the CPU 402 can be configured to control the energy delivery power supply 412, which in turn can supply power to the ablation energy source (e.g., the ablation energy source 103).

In an example, the algorithm 403 or the CPU 402 can be coupled to the retraction control 415. In an example, the retraction control 415 can be configured to control a retraction module (e.g., the retraction module 106). In an example, control of the retraction module can be fully-automated, partially automated, or at least partially user-controlled. In certain examples, the speed of the retraction can be controlled, or the length of the retraction can be controlled.

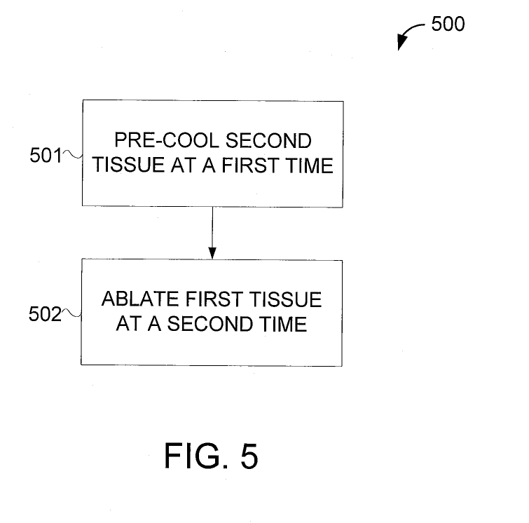

FIG. 5 illustrates generally an example of a method 500 including pre-cooling second tissue at a first time and ablating first tissue at a second time. In an example, the first tissue can include at least a portion of a vessel (e.g., at least a portion of a wall of the vessel), and the second tissue can include tissue proximate the first tissue, opposite a lumen of the vessel. In an example, the first tissue can include a first range of tissue extending outward from a lumen of the vessel, the first tissue including at least an inner wall of the vessel. The first range can terminate at or near a desired ablation depth. In an example, the second tissue can include a second range of tissue extending outward from the outer boundary of the first range. The second tissue can include tissue desired to be protected from ablation energy delivered to the first tissue.

At 501, the second tissue is pre-cooled at a first time. In an example, the first tissue and the second tissue can be pre-cooled at the first time. In an example, the second tissue can be pre-cooled using a cooling member (e.g., the expandable member 202).

At 502, the first tissue is ablated at a second time. In an example, the ablation energy is applied inside of a vessel. The ablation energy can extend into the first tissue and into the second tissue. As the distance from the ablation energy source increases, the effect of the ablation energy decreases. In an example, pre-cooling the second tissue can reduce the damaging effect of the ablation energy. In certain examples, some or all of the first tissue can be ablated, while some or all of the second tissue remains, at least in part due to the pre-cooling the second tissue.

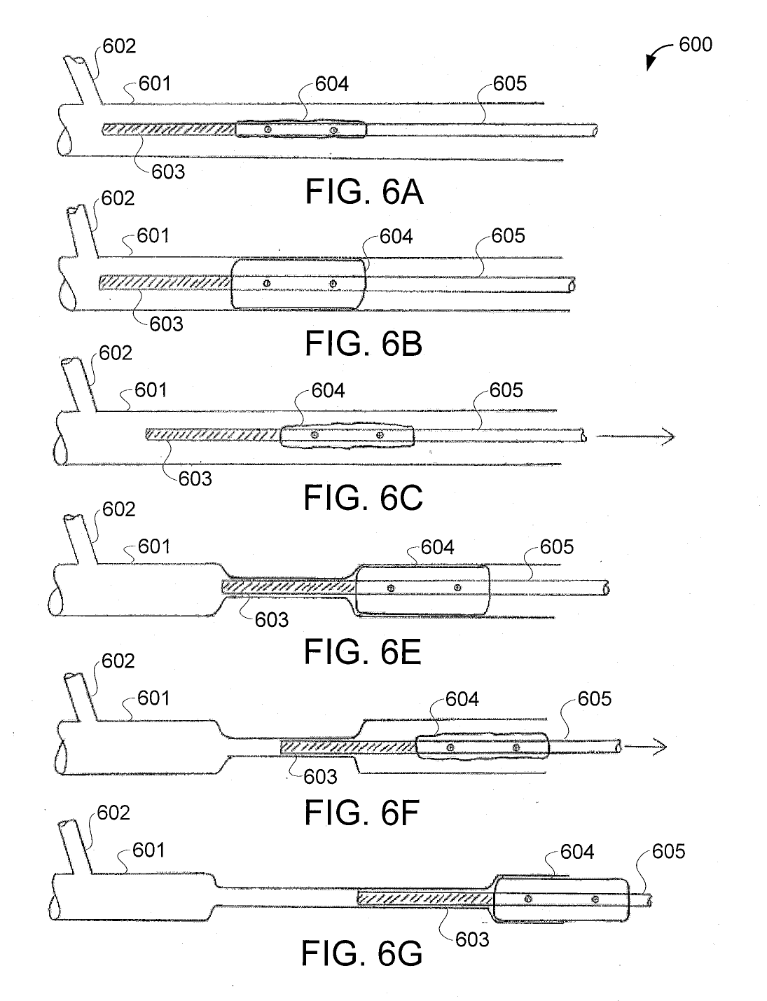

FIGS. 6A-6G illustrate generally examples 600 of pre-cooling and ablating at least a portion of a great saphenous vein 601. FIGS. 6A-6G include the great saphenous vein 601, a femoral vein 602 branching from the great saphenous vein 601, an ablation energy source 603, a cooling member 604, and an elongated tubular body 605.

At FIG. 6A, the elongated tubular body 605, the cooling member 604 (deflated), and the ablation energy source 603 are located inside the great saphenous vein 601 near the femoral vein 602. At FIG. 6B, the cooling member 604 is inflated (e.g., with a cooling liquid), and a first portion of the great saphenous vein 601 proximate the cooling member 604 is cooled. At FIG. 6C, after cooling the first portion of the great saphenous vein 601 for a period of time, or until the first portion of the great saphenous vein 601 or other tissue area reaches a predefined temperature (e.g., 30 degrees Celsius), cooling is stopped, and the cooling member 604 is deflated. After the cooling member 604 is deflated, the elongated tubular body 605 is retracted, so that the ablation energy source 603 is proximate the pre-cooled first portion of the great saphenous vein 601.

At FIG. 6E, the ablation energy source 603 delivers an ablation energy sufficient to shrink or ablate the pre-cooled first portion of the great saphenous vein 601. As the pre-cooled first portion of the great saphenous vein 601 is ablated, at least a portion of the tissue proximate the ablated first portion is left undamaged, or is left having less damage than it would have otherwise received due at least in part to the pre-cooling. Further, at FIG. 6E, as the ablation energy source 603 is moved proximate the first portion of the great saphenous vein 601, the cooling member 604 is moved proximate a second portion of the great saphenous vein 601. The cooling member 604 is inflated (e.g., with the cooling liquid), and the second portion of the great saphenous vein 601 proximate the cooling member 604 is cooled.

At FIG. 6F, after ablating the first portion of the great saphenous vein 601 for a period of time, or until the first portion of the great saphenous vein 601 or other tissue area reaches a predefined temperature (e.g., 70 degrees Celsius for tissue to be ablated, 37 degrees Celsius for tissue not to be ablated, etc.). Further, at FIG. 6F, after cooling the second portion of the great saphenous vein 601 for a period of time, or until the first portion of the great saphenous vein 601 or other tissue area reaches a predefined temperature (e.g., 30 degrees Celsius), cooling is stopped, and the cooling member 604 is deflated. In certain examples, the ablating at the first portion and the cooling at the second portion can be done at the same or different times.

At FIG. 6G, the ablation energy source 603 delivers an ablation energy sufficient to shrink or ablate the pre-cooled second portion of the great saphenous vein 601. As the pre-cooled second portion of the great saphenous vein 601 is ablated, at least a portion of the tissue proximate the ablated first portion is left undamaged, or is left having less damage than it would have otherwise received due at least in part to the pre-cooling. Further, at FIG. 6G, as the ablation energy source 603 is moved proximate the first portion of the great saphenous vein 601, the cooling member 604 is moved proximate a third portion of the great saphenous vein 601. The cooling member 604 is inflated (e.g., with the cooling liquid), and the third portion of the great saphenous vein 601 proximate the cooling member 604 is cooled.

In certain examples, this process can be repeated until a desired portion of the great saphenous vein 601 or other vessel is sufficiently ablated. In certain examples, the movement or retraction, such as between the first portion and the second portion of the great saphenous vein 601, can be performed using a retraction module (e.g., the retraction module 113).

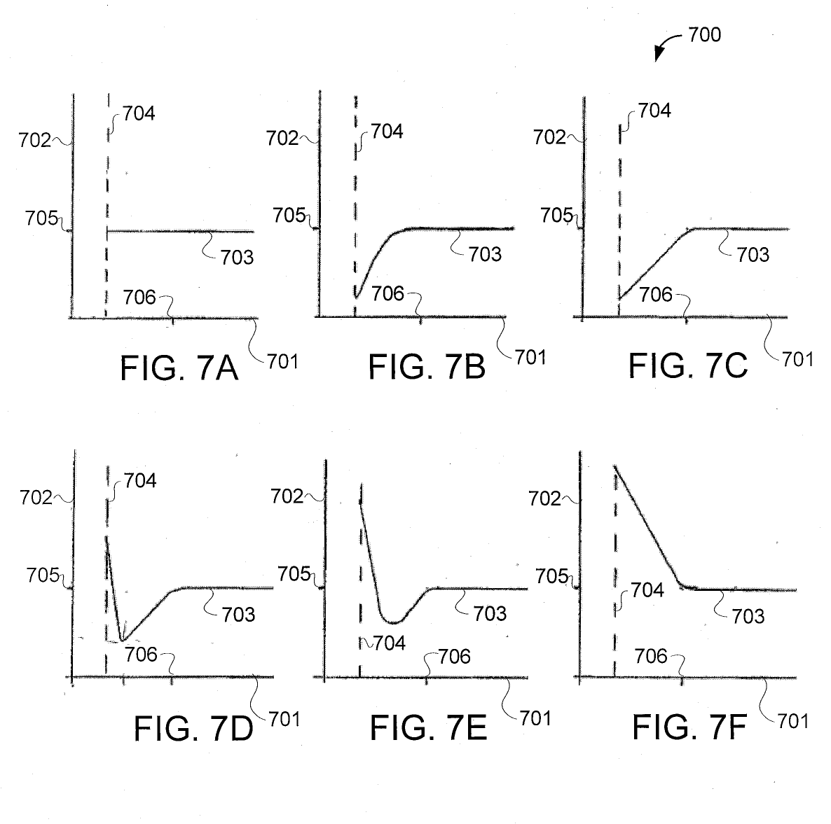

FIGS. 7A-7F illustrate generally examples (not real data) of expected relationships 700 between temperature 702 and distance 701 for a vessel and surrounding tissue during pre-cooling and ablation. In FIGS. 7A-7F, the origin of the distance 701 indicates a center of the vessel, with a vessel inner wall 704 being marked along the distance 701 axis. Tissue temperature 703 varies with heating and cooling along the distance 701 axis. Desired ablation depth 706 is marked along the distance 701 axis. In certain examples, the region between the vessel inner wall 704 and the desired ablation depth 706 can include a first tissue region, where ablation is desired. The region extending outward from the desired ablation depth 706 can include a second tissue region, where ablation is not desired. In these examples, a normal tissue temperature 705 (e.g., 37 degrees Celsius) is indicated along the temperature 702 axis.

At FIG. 7A, no cooling or ablation is taking place, and the tissue temperature 703 remains at the normal tissue temperature 705. At FIG. 7B, pre-cooling begins. Accordingly, in this example, the tissue temperature 703 is lower towards the vessel inner wall 704, due to the cooling. At FIG. 7C, pre-cooling is complete or near-complete, as the tissue temperature 703 remains relatively linear between the vessel inner wall 704 and the desired ablation depth 706.

At FIG. 7D, ablation begins. In this example, the tissue temperature 703 is highest at the vessel inner wall 704 (e.g., 70 degrees Celsius), and falling until it meets the previously established pre-cooling temperature before the desired ablation depth 706. At FIG. 7E, ablation continues, and the tissue temperature 703 further climbs towards the normal tissue temperature 705 throughout the region between the vessel inner wall 704 and the desired ablation depth 706. At FIG. 7F, ablation is complete or near-complete, as the tissue temperature 703 remains relatively linear between the vessel inner wall 704 and the desired ablation depth 706.

In these examples, the tissue temperature 703 does not change substantially from the normal tissue temperature 705 after the desired ablation depth 706. If no pre-cooling were to occur, then tissue temperature 703 would be above the normal tissue temperature 705 deeper into the tissue.

### Other Examples

In an example, external cooling of the leg or other area surrounding the vessel can be used in combination with the pre-cooling.

In other examples, cooling can be continued following the ablation (e.g., post-cooling) to further assist in reducing tissue damage or discomfort or pain associated with tissue damage. In various examples, a second cooling member located distal to an ablation energy source, having a smaller diameter than a first cooling member or ablation energy source, can be used to reduce the damage to the ablated areas.

In other examples, the ablation energy source can include a stepped region, such that a first area can be pre-cooled at a first time, a first ablation energy with the first stepped region can be applied at a second time, and a second ablation energy with the second smaller stepped region can be applied and a third time.

In certain examples, control of the ablation energy delivery can include open loop control (e.g., time period) or closed loop control (e.g., temperature or other feedback, such as electrical impedance if an electric energy (e.g., current) ablation delivery method is utilized, etc.).

In an example, cooling a first area can be done simultaneous to, overlapping in time with, or sequentially with ablating a second area.

### Example of a Procedure

In an example, one or more procedures can be used such as in accordance with the examples disclosed herein. One example of a procedure is illustrated below:

### Sterile

(1) Provide a sterile work area, including sterilizing a localized skin area near a knee and draping off the remainder of the leg to provide a sterile work area.

(2) Provide venous access (e.g., using Seldinger technique) to insert an access sheath at a desired location. (Optional: Deliver guide wire into saphenous vein.)

(3) Prepare the therapeutic device.
a. Flush the guide wire port (e.g., using saline).
b. Remove the air (De-air) from the balloon

(4) Insert the therapeutic device via the access sheath into the saphenous vein and advance the therapeutic device to the femoral vein.

(5) Attach the proximal portion of therapeutic device to a retraction module.

### Non-sterile

(6) Identify critical features at the sapheno-femoral junction using external ultrasound.

(7) Retract the therapeutic device such that the distal end of the balloon is adjacent to the most cranial portion of the vein to be treated.

(8) Initiate inflation of the balloon and circulate cooling fluid for desired time or to a desired temperature.

(9) Deflate the balloon.

(10) Retract the therapeutic device to move the energy delivery portion of the device adjacent to the most cranial portion of the vein to be treated.

(11) Apply energy to treat the vein, limiting the energy delivery by time or temperature feedback to prevent inadvertent thermal damage to tissues adjacent the target vein.

(12) Repeat acts 8 through 11 until the desired length of the vein is treated.

(13) Optional: A single segment of vein can be treated multiple times to achieve complete shrinkage or coagulation. Here, the catheter may be advanced back to the starting location, the tissue re-cooled, and thermally treated again. Ultrasound can be used to monitor the vein to ensure adequate treatment prior to retracting the device entirely from the treatment zone.

In certain examples, acts 8 through 12 can be controlled partially or completely by a computer (e.g., an actuator) and associated algorithm to ensure repeatable, consistent, and effective treatment without the need for user intervention, tuning, or judgment.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system for ablating first tissue, including pre-cooling nearby second tissue to inhibit damage to the second tissue resulting from the ablation, the system comprising:
a cooling member located near a distal end of an elongated tubular body configured to be inserted into a vessel, the cooling member configured to pre-cool the second tissue;
an ablation energy source located near the cooling member, the energy source configured to provide an ablation energy to the first tissue; and
an actuator configured to actuate the pre-cooling and the ablation such that the pre-cooling occurs before the ablation.

2. The system of claim 1, including the elongated tubular body, wherein the elongated tubular body includes a lumen configured to deliver a cooling matter to the cooling member, the cooling matter including at least one of a fluid or a gas.

3. The system of claim 2, wherein the cooling member includes an expandable member, wherein the actuator is configured to actuate inflation of the expandable member, using the cooling matter, the cooling matter including at least one of a chilled fluid, a chilled gas, or an expanding pressurized gas.

4. The system of claim 1, wherein the ablation energy source configured to provide the ablation energy includes at least one of an electrode configured to provide a radiofrequency energy, a first and second electrode configured to provide and to receive an electric current, an ultrasound transducer configured to provide an ultrasound energy, or an optical element configured to provide a laser energy.

5. The system of claim 1, wherein the actuator includes at least one of a user-initiated actuator configured to receive an actuation instruction from a user or an automated actuator configured to automatically provide an actuation instruction.

6. The system of claim 1, including a temperature sensor configured to receive a temperature of at least one of the first tissue, the second tissue, or the ablation energy source; and
wherein the actuator is configured to receive information from the temperature sensor, and to actuate at least one of the pre-cooling or the ablation using the received temperature information.

7. The system of claim 1, wherein the first tissue includes at least a portion of a wall of the vessel, the vessel including at least one of a great saphenous vein or a small saphenous vein, and wherein the second tissue includes tissue proximate the first tissue, opposite a lumen of the vessel.

8. The system of claim 1, including:
the elongated tubular body; and
a retraction module configured to at least partially retract the elongated tubular body from the vessel, wherein the actuator is configured to actuate the retraction.

9. The system of claim 8, wherein the actuator is configured to actuate the pre-cooling the second tissue at a first time at a first location, wherein the actuator is configured to retract the cooling member from the first location to a second location and the ablation energy source to the first location following the pre-cooling, and wherein the actuator is configured to actuate the ablating the first tissue at a second time at the first location, the second time later than the first time.

10. The system of claim 9, wherein the cooling member is configured to pre-cool the second tissue at the first time to inhibit damage to the second tissue at the second time.

11. The system of claim 1, wherein the damage resulting from the ablation includes at least one of thermal damage, or pain or discomfort associated with the thermal damage.

12. A method for ablating first tissue and inhibiting damage to nearby second tissue resulting from the ablation, comprising:
pre-cooling the second tissue at a first time;
ablating the first tissue at a second time later than the first time; and
wherein the pre-cooling the second tissue at the first time inhibits damage to the second tissue at the second time resulting from the ablation.

13. The method of claim 12, wherein the pre-cooling includes delivering a cooling matter to a cooling member located near a distal end of an elongated tubular body configured to be inserted into a vessel, and wherein the delivering the cooling matter includes delivering at least one of a chilled fluid or a chilled gas.

14. The method of claim 13, wherein the pre-cooling includes inflating an expandable member using the cooling matter, the cooling matter including at least one of a chilled fluid, a chilled gas, or an expanding pressurized gas.

15. The method of claim 12, wherein the ablating includes using an ablation source, the ablation source providing at least one of a radiofrequency energy, an electric current, an ultrasound energy, or a laser energy.

16. The method of claim 12, including at least one of:
receiving a pre-cooling instruction, configured to control the pre-cooling, and an ablating instruction, configured to control the ablating, from a user-initiated actuator; or
automatically controlling the pre-cooling and the ablating using an automated actuator.

17. The method of claim 12, including receiving temperature information about a temperature of at least one of the first tissue, the second tissue, or the ablation energy source; and
wherein the pre-cooling the second tissue and the ablating the first tissue includes using the received temperature information.

18. The method of claim 12, wherein the ablating the first tissue includes ablating at least a portion of a wall of a vessel, the vessel including at least one of a great saphenous vein or a small saphenous vein, and wherein the pre-cooling the second tissue includes tissue proximate the first tissue, opposite a lumen of the vessel.

19. The method of claim 12, wherein the pre-cooling includes pre-cooling the second tissue at a first location at the first time using a cooling member located near a distal end of an elongated tubular body configured to be inserted into a vessel, wherein the method includes:
at least partially retracting the elongated tubular body from the first position to a second location using an automated retraction module; and
wherein the ablating includes ablating the first tissue at the first location at the second time using an ablation energy source located near the cooling member.

20. The method of claim 12, wherein the pre-cooling includes inhibiting damage to the second tissue resulting from the ablation at the second time, wherein the damage includes at least one of thermal damage, or pain or discomfort associated with the thermal damage.
